(19) United States
(12) Patent Application Publication
(10) Pub. No.: US 2012/0143134 A1
(43) Pub. Date: Jun. 7, 2012

(54) Method of Performing Laparoscopic Surgery Using a Multi-Access Channel Surgical Trocar

(76) Inventors: Jeffrey D. HOLLIS, Gallatin, TN (US); Gregory D. HOLLIS, Nashville, TN (US)

(21) Appl. No.: 13/154,171

(22) Filed: Jun. 6, 2011

Related U.S. Application Data
(63) Continuation-in-part of application No. 11/941,258, filed on Nov. 16, 2007, now abandoned.

(51) Int. Cl. A61B 17/34

(52) U.S. Cl. 604/164.01

(57) ABSTRACT

The present invention provides a method of performing laparoscopic surgery using a surgical trocar including an elongated trocar passageway and an access channel disposed in spaced relationship to each other. The access channel includes a gas seal to prevent gas leakage while the access channel is in use, and a valve to close the access channel when not in use. Once, the trocar is inserted into the body cavity, the access channel allows access of catheters and or other small diameter devices without the need for making extra incisions. This allows continued use of the trocar while utilizing the access channel, giving it a dual function usage. The access channel allows devices to be angled away from the trocar once inside the body cavity making device manipulation easier.

### CROSS REFERENCE TO RELATED APPLICATIONS

This application is a Continuation-In-Part and contains disclosure from and claims the benefit under Title 35, United States Code, §120 of the following U.S. Non-Provisional Patent Application: U.S. application Ser. No. 11/941,258 filed Nov. 16, 2007, entitled SURGICAL TROCAR, and is incorporated in its entirety.

### BACKGROUND OF THE INVENTION

1. Field of the Invention

The present invention relates to the field of surgical methods, and more particularly to a method of performing laparoscopic surgery using a surgical trocar having multiple access channels disposed in spaced relationship to each other.

2. Description of Related Art

As can be seen by reference to the following U.S. Pat. Nos. 5,139,487; 5,743,881; 5,941,852; U.S. Publn. 20070016100; and EP0756505, the prior art is replete with myriad and diverse surgical trocars.

While all of the aforementioned prior art constructions are adequate for the basic purpose and function for which they have been specifically designed, they are uniformly deficient with respect to their failure to provide a simple, efficient, and practical trocar having an integrated access channels to be used in performing laparoscopic surgery.

As a consequence of the foregoing situation, there has existed a longstanding need for a new and improved surgical method, and the provision of such a method is a stated objective of the present invention.

### BRIEF SUMMARY OF THE INVENTION

Briefly stated, the present invention provides a method of performing laparoscopic surgery using a surgical trocar including an elongated trocar passageway and an access channel disposed in spaced relationship to each other. The access channel includes a gas seal to prevent gas leakage while the access channel is in use, and a valve to close the access channel when not in use. Once, the trocar is inserted into the body cavity, the access channel allows access of catheters and or other small diameter devices without the need for making extra incisions. This allows continued use of the trocar while utilizing the access channel, giving it a dual function usage. The access channel allows devices to be angled away from the trocar once inside the body cavity making device manipulation easier.

One embodiment of the trocar has the access channel integrally formed in a housing with the trocar passageway. Another embodiment of the trocar includes a sheath that receives the elongated housing of a conventional trocar where the sheath includes an access channel disposed in spaced relationship to the trocar passageway.

The objective of this invention is to provide a multipurpose access channel for a trocar, for use during laparoscopic surgery. Current laparoscopy trocars do not provide an access channel and thus require separate incisions if a small diameter device, such as a catheter, is to be used. This improves current trocars by making them more versatile and eliminates the need for excess incisions Currently, during laparoscopic cholecystectomy, a separate stab incision is required to insert a cholangiocatheter or stone retrieval device. This new access channel provides integrated access through the existing trocar, removing the need for extra incisions.

### BRIEF DESCRIPTION OF THE SEVERAL VIEWS OF THE DRAWINGS

These and other attributes of the invention will become more clear upon a thorough study of the following description of the best mode for carrying out the invention, particularly when reviewed in conjunction with the drawings, wherein:

FIG. 1 is a side elevational view of the surgical trocar of the present invention, with the proximal and distal ends cut away;

FIG. 2 is an enlarged partial sectional view of the proximal end of the trocar;

FIG. 3 is an enlarged partial sectional view of the distal end of the trocar;

FIG. 4 is a proximal end elevational view of the trocar;

FIG. 5 is a distal end elevational view of the trocar;

FIG. 6 is a side elevational view of a glove sheath for a conventional trocar, with the proximal and distal ends cut away;

FIG. 7 is an enlarged partial sectional view of the proximal end of the sheath;

FIG. 8 is an enlarged partial sectional view of the distal end of the sheath;

FIG. 9 is a proximal end elevational view of the sheath;

FIG. 10 is a distal end elevational view of the sheath; and

FIGS. 11A through 11D are schematic representations showing the trocar of the present invention in use to perform laparascopic cholecystectomy.

### DETAILED DESCRIPTION OF THE INVENTION

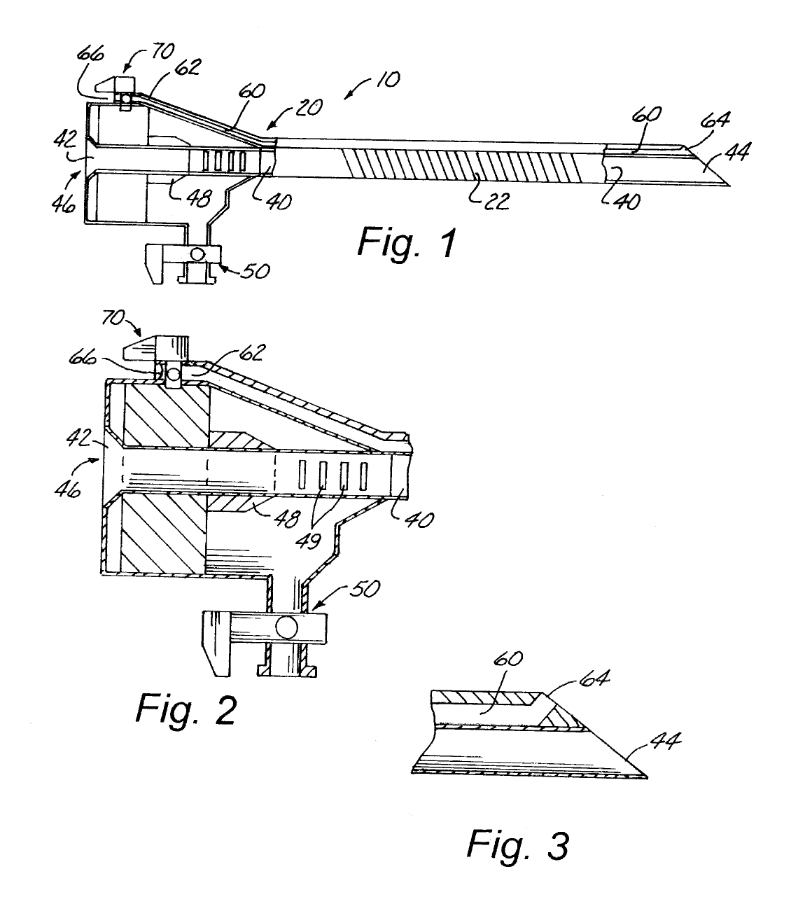

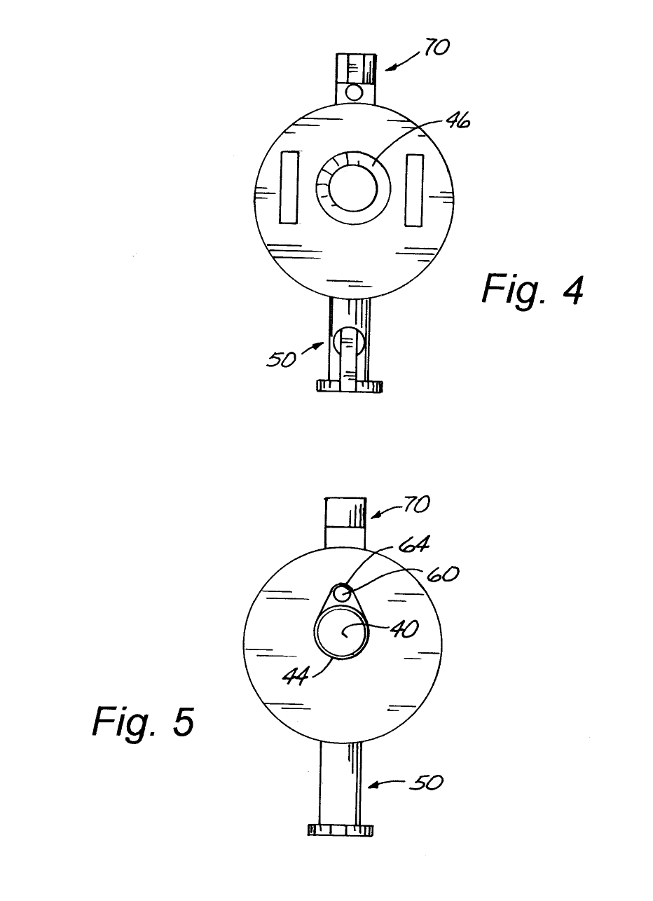

As can be seen by reference to the drawings, and in particular to FIG. 1, the surgical trocar used in the method of the present invention is designated generally by the reference number 10. The trocar 10 includes an elongated housing 20 with a trocar passageway 40 and an access channel 60 formed in the housing 20 in spaced relationship to each other. The exterior of the housing 20 has grooves 22 for tissue traction.

The trocar passageway 40 has a proximal end 42 and a distal end 44. The proximal end 42 is in open communication with the trocar collar 46 which includes a rubber boot gas seal 48 through which the instrument (not shown) is directed and internal gas inlets 49. A stopcock valve 50 is also in communication with the trocar passageway 40. As shown in FIGS. 1 and 3, the open distal end 64 of the access channel 60 and the open distal end 44 of the trocar passageway 40 are aligned along the same plane which intersects the longitudinal axis of the trocar passageway 40. The intersecting plane is disposed at an acute angle of about 45° with respect to the longitudinal axis.

The access channel 60 has a proximal end 62 and a distal end 64. A diaphragm gas seal 66 is disposed near the proximal end 62 to prevent gas leakage out through the access channel 60 when it is in use. Also, a stopcock valve 70 is provided to close the access channel 60 when it is not in use. A portion of the access channel 60 at the distal end 64 is directed away from the longitudinal axis of the trocar passageway 40 so that devices carried through the access channel 40 may be easily manipulated without interference with the instrument introduced through the trocar passageway 40. As shown in FIGS. 1 and 3, the open distal end 64 of the access channel 60 and the open distal end 44 of the trocar passageway 40 are aligned along the same plane which intersects the longitudinal axis of the trocar passageway 40. The intersecting plane is disposed at an acute angle of about 45° with respect to the longitudinal axis.

The invention may be used for different small diameter devices on different size trocars. The trocar function itself is not compromised. This access channel 60 can be used for diagnostic and therapeutic interventions depending on the device. Diagnostic uses, for example, would include a channel for cholangiography or liver biopsy. Therapeutic uses, for example, would include catheters for bile duct exploration such as stone baskets.

The trocar is used for laparoscopic surgery, wherein minimally invasive incisions are created to perform operations inside the abdomen. Typically a “trocar” is used as the portal/conduit for passage of instruments while performing these operations. During laparoscopic surgery, a number of “port's” or “trocars” may be used based on the difficulty and complexity of the procedure.

The method of the present invention describes the use of a trocar with multiple channels which further minimizes the number of portals needed for a specific surgery. In laparoscopic gallbladder surgery (laparoscopic cholecystectomy) there is often the need to perform an intra-operative cholangiogram (IOC). The goal is to evaluate the bile duct system for anatomy and for the presence of gallstones that may have escaped from the gallbladder itself. This is important to prevent injury to vital structures and to identify potential problems of obstruction from gallstones that may have escaped the gallbladder into the biliary ductal system. To perform a cholangiogram, a catheter is inserted (usually percutaneously via a separate incision) into the cystic duct. This allows for the infusion of radio-opaque dye into the biliary system which can be visualized by fluoroscopy to accomplish the above mentioned goals. Further interventions and operative plans can then be made based on those findings. The importance of the multi-access trocar is to minimize the number of incisions made, maximize patient safety, maximize trocar efficiency, and shorten operative times.

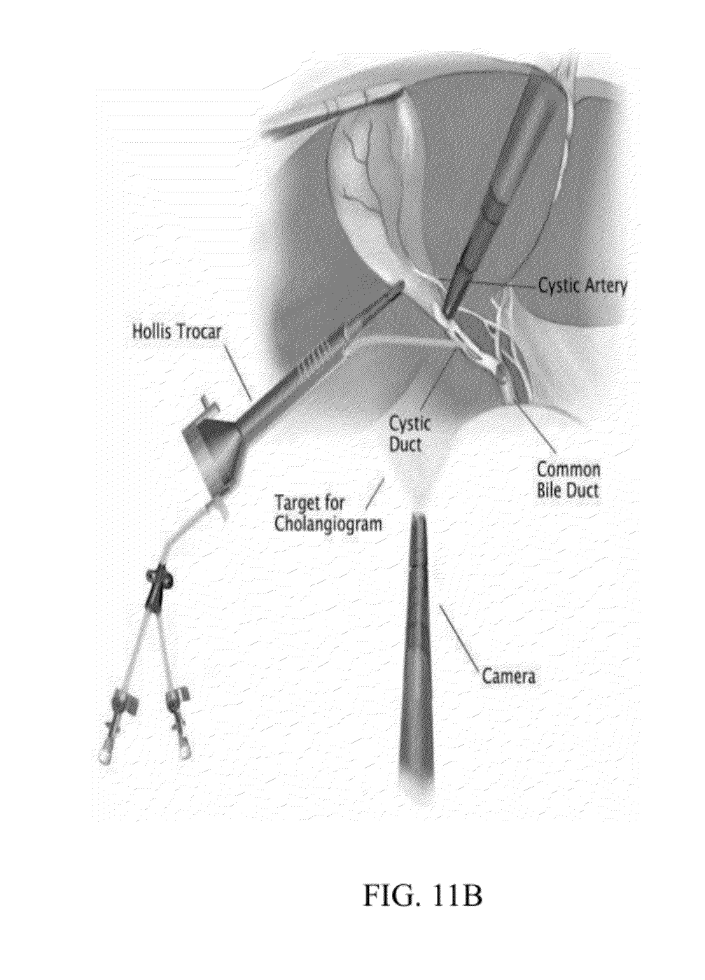

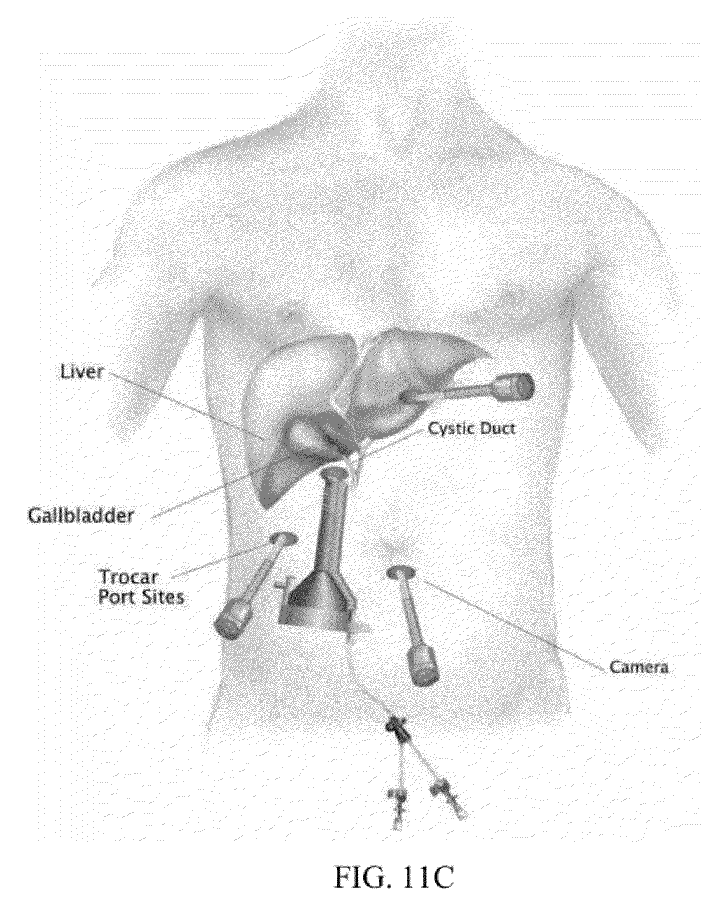

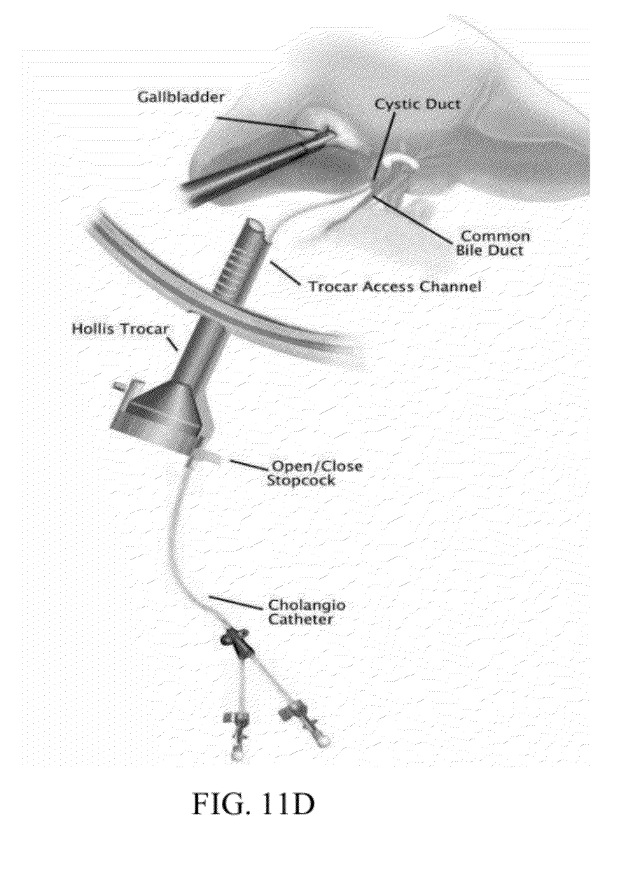

The laparascopic cholecystectomy is generally illustrated in FIGS. 11A through 11D. Specifically, the method (once the patient is in the O.R.) includes the following steps:
1. correctly position the patient;
2. prep the abdomen;
3. insert multi-access trocars in locations based on anatomy;
4. visually explore abdomen;
5. identify and retract the gallbladder;
6. dissect and isolate the cystic duct;
7. place 2 clips on proximal cystic duct;
8. make a small ductotomy in the cystic duct;
9. open access channel on trocar and insert cholangiocatheter through access channel on the multi-access trocar and place into the cystic duct;
10. inflate stay balloon or clip cholangiocatheter in place in the cystic duct;
11. infuse radio-opaque dye through cholangiocatheter and into biliary system;
12. evaluate radiographic findings;
13. intervene as necessary;
14. deflate stay balloon or remove clip from cholangiocatheter;
15. remove cholangiocatheter from access channel and close channel;
16. finish surgical removal of gallbladder in usual fashion. Access channel may be opened as needed for smoke evacuation during operation;
17. irrigate abdomen as needed;
18. remove all multi-access trocars;
19. suture skin sites closed; and
20. place dressings.

Basically the method includes the steps of inserting the trocar, opening the channel, using the channel, closing the channel, and removing the trocar.

The trocar may be structured differently by changing the location of the access channel 60 either longitudinally or obliquely in reference to the trocar passageway 40. The access channel 60 can be structured to different diameters to accommodate different interventional devices. The opening of the channel 60 can be structured to open at the distal end 44 or anywhere along the trocar passageway 40. The trocar itself can be structured to different sizes depending on what access to a body cavity is needed. Multiple access channels can be placed on a single trocar. The channel can be incorporated into either cutting or dilating type trocars. The cross section of the housing 20 could be modified from the tear drop shape shown in FIG. 5 without changing the functionality.

The device works by not only allowing laparoscopic access to a body cavity, but also by providing a built in channel 60 for small diameter device access. The trocar passageway 40 allows laparoscopy instruments to be inserted into a body cavity for surgery. The integrated access channel 60 allows simultaneous use of the trocar 10 for other small diameter devices, such as cholangiocatheters, without compromising the use of the trocar passageway 40 for instruments. This design provides a versatile alternative to current trocars.

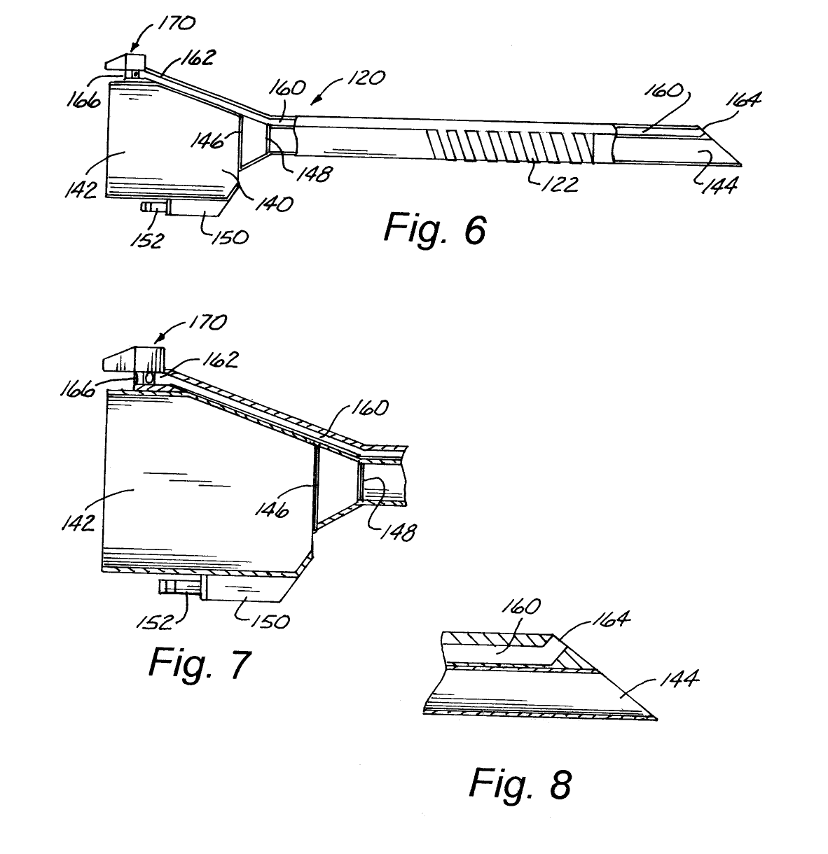

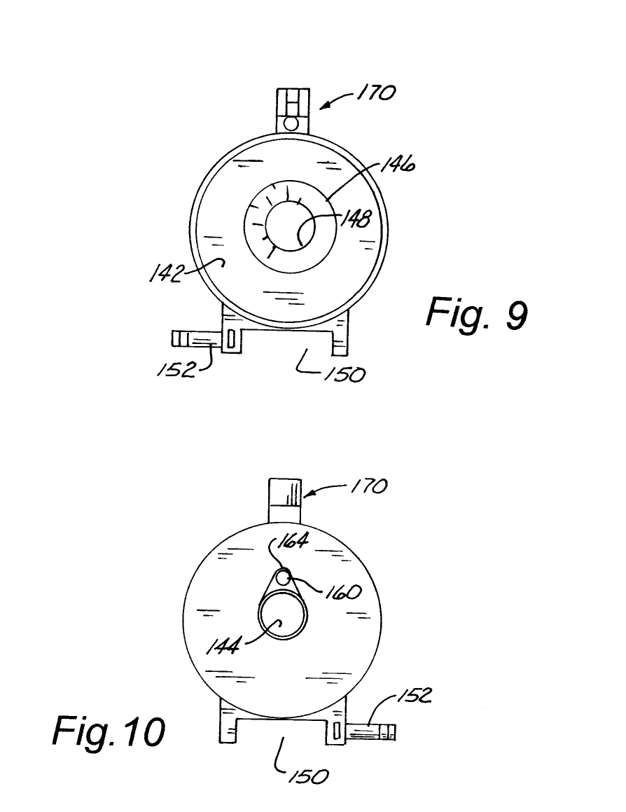

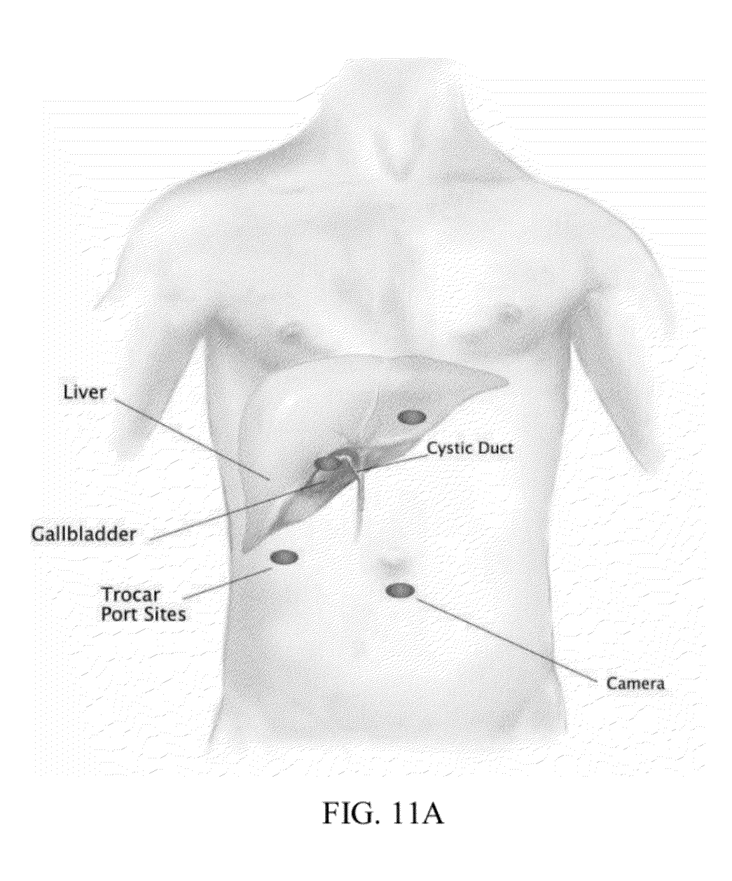

A second embodiment of the trocar uses a glove sheath 120 as shown in FIGS. 6-10, in combination with a conventional trocar. The sheath 120 is disposed to matingly receive the housing of a conventional trocar. The sheath 120 includes a trocar receiving cavity 140 and an access channel 160 disposed in spaced relationship to the cavity 140. The exterior of the sheath 120 has grooves 122 for tissue traction.

The trocar cavity 140 has a proximal end 142 and a distal end 144. A pair of “O”-ring seals 146 and 148 prevents gas leakage and stabilizes the trocar within the cavity 140. A groove 150 receives the air insufflation port of the trocar, and a latch 152 secures the trocar within the sheath 120 at the air insufflation point.

The access channel 160 has a proximal end 162 and a distal end 164. A diaphragh gas seal 166 and a stopcock valve 170 are carried near the proximal end 162. The sheath 120 thus provides a retrofit of a conventional trocar that functions like the main embodiment of the present invention. FIGS. 6 and 8 show the relationship of the open distal ends 164 and 144 of the access channel 160 and the trocar passageway 140 of this second embodiment, where the distal ends 164 and 144 are aligned along the same plane that intersects the longitudinal axis of the trocar passageway 140 at an acute angle of approximately 45°.

Although only an exemplary embodiment of the invention has been described in detail above, those skilled in the art will readily appreciate that many modifications are possible without materially departing from the novel teachings and advantages of this invention. Accordingly, all such modifications are intended to be included within the scope of this invention as defined in the following claims.

Having thereby described the subject matter of the present invention, it should be apparent that many substitutions, modifications, and variations of the invention are possible in light of the above teachings. It is therefore to be understood that the invention as taught and described herein is only to be limited to the extent of the breadth and scope of the appended claims.

I claim:

1. A method of performing laparoscopic surgery, the method comprising the steps of:
providing a surgical trocar having an elongated housing having a proximal end and a distal end, the trocar including an elongated trocar passageway and a separate elongated access channel disposed in spaced relationship to each other;
making a single incision providing access to a body cavity of a patient;
inserting the distal end of the housing through the single incision such that the access channel are disposed within the body cavity; and
simultaneously inserting a laparoscopy instrument into the body cavity through the trocar passageway, and inserting a device into the body cavity through the access channel.

2. The method of claim 1, wherein the device is selected from a group consisting of diagnostic devices and therapeutic devices.

3. The method of claim 2, wherein the device is a catheter.

4. The method of claim 1, wherein the laparoscopic surgery is a laparoscopic cholecyotectomy including the steps of:
correctly positioning the patient;
prepping the abdomen;
inserting the surgical trocar in a location based on anatomy;
visually exploring the abdomen;
identifying and retracting the gallbladder;
dissecting and isolating the cystic duct;
placing two (2) clips on the proximal cystic duct;
making a small ductotomy in the cystic duct;
opening the access channel on the trocar and inserting a cholangiocatheter through the access channel and placing the cholangiocatheter into the cystic duct;
inflating a stay balloon or a clip cholangiocatheter in place in the cystic duct;
infusing radio-opaque dye through the cholangiocatheter and into the biliary system;
evaluating radiographic findings;
intervening as necessary;
deflating the stay balloon or removing the clip from the cholangiocatheter;
removing the cholangiocatheter from the access channel and closing the access channel;
finishing surgical removal of the gallbladder in the usual fashion;
opening the access channel as needed for smoke evacuation during the operation;
irrigating the abdomen as needed;
removing the trocar;
suturing the skin sites closed; and
placing dressings.
